(19) United States
(12) Patent Application Publication
(10) Pub. No.: US 2005/0187560 A1
(43) Pub. Date: Aug. 25, 2005

(54) Apparatus and method for sizing a distal femur

(76) Inventors: Steven E. Dietzel, Peru, IN (US); Toby N. Farling, Warsaw, IN (US); Adam M. Griner, Columbia City, IN (US); Robert A. Hodorek, Warsaw, IN (US); W. Matthew Kuester, Fort Wayne, IN (US); Vincent A. Webster, Warsaw, IN (US); Alyssa M. Perry, Winona Lake, IN (US); Linggawati Tanamal, Singapore (SG)

Correspondence Address: ZIMMER TECHNOLOGY - ROBERTS;INDIANO, VAUGHN & ROBERTS, 1 NORTH PENNSYLVANIA AVENUE #850, INDIANAPOLIS, IN, 46204, US

(21) Appl. No.: 11/047,205

(22) Filed: Jan. 28, 2005

Related U.S. Application Data
(60) Provisional application No. 60/540,616, filed on Jan. 29, 2004.

(51) Int. Cl. A61B 17/58

(52) U.S. Cl. 606/102

(57) ABSTRACT

An apparatus for sizing a distal femur includes a base configured to concurrently abut a distal surface and a posterior surface of the distal femur, and further includes an axial distance scale. The scale includes a first sleeve axially fixedly coupled to the base and further includes a second sleeve spirally movably coupled to the first sleeve. A method for sizing a distal femur includes concurrently abutting a base against a distal surface and a posterior surface of the distal femur, and further includes measuring a distance relative to the base. The measuring step includes spirally moving a first sleeve relative to a second sleeve.

### CROSS-REFERENCE TO RELATED APPLICATION(S)

This application claims the benefit of U.S. Provisional Patent Application Ser. No. 60/540,616, filed Jan. 29, 2004, entitled “APPARATUSES AND METHODS FOR ARTHROPLASTIC. SURGERY.”

### FIELD OF THE INVENTION

The present invention relates generally to the field of orthopaedics, and, more particularly, to an apparatus and method for sizing a distal femur.

### BACKGROUND

Total joint arthroplasty (“joint replacement”) is the surgical replacement of a joint with a prosthesis. A typical knee prosthesis has three main components: a femoral implant, a tibial implant, and a tibio-femoral insert. In general, the femoral implant is designed to replace the distal femoral condyles. The femoral implant is typically made from metal. It typically includes a generally concave, facetted (i.e., piecewise planar) inwardly facing surface defining a cavity for receiving a resected distal femur and typically further includes a generally convex outwardly facing surface with medial and lateral rounded portions for emulating the medial and lateral condyles, respectively, and with a valley or depression between the rounded portions for emulating the patella sulcus/trochlear region of the distal femur. In general, the tibial implant is designed to support and align the tibio-femoral insert. The tibial implant is also typically made from metal. It typically includes a substantially planar tray or plate portion (“tibial plate”) for supporting the insert, and an elongated stem extending away from the tibial plate for anchoring the tibial implant in the intramedullary canal of the proximal tibia. In general, the tibio-femoral insert is designed to replace the tibial plateau and the meniscus of the knee. It is typically somewhat disk-shaped, and typically includes one or more substantially planar surfaces for bearing on the tibial plate and one or more generally concave surfaces for bearing against the femoral implant. The insert is typically made of a strong, smooth, low-wearing plastic.

In a conventional knee replacement operation, the surgeon makes an anterior incision spanning the distal femur, the knee, and the proximal tibia; everts (i.e., flips aside) the patella; separates the distal femur and the proximal tibia from surrounding tissues; and then hyperflexes, distally extends, and/or otherwise distracts the proximal tibia from the distal femur to enlarge the operating space. Next, the surgeon secures a resection guide to the proximal tibia. A resection guide is a jig or template configured to provide a desired cutting angle for a saw blade or other resection tool. Conventional resection guides are used somewhat similarly to the manner in which a carpenter uses a miter box to achieve a desired angle for cutting wood. The surgeon uses the resection guide to position to a saw blade or other suitable resection tool and cuts off the tibial plateau. This prepares the proximal tibia to receive the tibial implant. Then, the surgeon aligns one or more additional resection guides for cutting and shaping the distal femur as required to fit the femoral implant. Resection of the distal femur typically includes removing anterior portions of the medial and lateral condyles. The position of the corresponding anterior cut is typically referenced or measured-off from posterior surfaces of the condyles. The distance from the posterior surfaces to the plane of the anterior cut typically corresponds to an anterior-posterior size for the receiving cavity of the femoral implant. Femoral implants are typically available only in a limited number of predetermined sizes. Accordingly, the surgeon typically attempts to limit the bone removed to that which must be disposed of due to deterioration plus the minimum additional amount required to accommodate the closest standard sized implant. After completing the necessary resections, the surgeon may apply cement to the distal femur and/or to the proximal tibia to ultimately help hold the femoral implant and/or tibial implant, respectively, in place. Alternatively, cementless implants may be used. Finally, the surgeon secures the respective implants to the distal femur and proximal tibia, secures the cushion to the top of the tibial implant, and closes the incision. If the operation is successful, the artificial knee properly mimics the operation of a healthy natural knee.

Complications may result if either the distal femur or the proximal tibia is not resected properly. Such complications can include accelerated wear of the prosthesis; bending, cracking or fracture of the remaining parts of the proximal tibia and/or distal femur; dislocation of the prosthesis, excessive rotation or loss of motion of the prosthesis; and/or angular deformity of the prosthetic joint.

Some devices have included features for positioning anterior cuts to the distal femur. Other devices have included features for indicating corresponding anterior-posterior femoral implant sizes. However, maintaining accurate correlations between separate mechanisms for positioning the cuts and indicating the implant sizes has been challenging. Preventing undesirable slippage in such mechanisms during operations has also been challenging.

Moreover, minimally invasive surgical techniques are becoming increasingly popular. Minimally invasive surgeries generally involve, among other things, considerably smaller incisions and tighter working spaces than historical techniques in efforts to reduce patient traumas and accelerate post-operative recoveries. Some devices for positioning anterior cuts to the distal femur and/or for indicating corresponding anterior-posterior femoral implant sizes are not well suited for operation in the tight spaces of minimally invasive surgeries.

### SUMMARY OF THE INVENTION

The present invention provides an apparatus for sizing a distal femur having a distal surface located in a first plane and a posterior surface located in a second plane angularly disposed from the first plane by about 90 degrees. The apparatus includes a base configured to concurrently abut the distal surface and the posterior surface, and further includes an axial distance scale. The scale includes a first sleeve axially fixedly coupled to the base and further includes a second sleeve spirally movably coupled to the first sleeve.

The present invention provides an apparatus for sizing a distal femur having a distal surface located in a first plane and a posterior surface located in a second plane angularly disposed from the first plane by about 90 degrees. The apparatus includes means for concurrently abutting the distal surface and the posterior surface, and further includes means, coupled to the abutting means, for measuring a distance relative to the abutting means.

The present invention provides a method for sizing a distal femur having a distal surface located in a first plane and a posterior surface located in a second plane angularly disposed from the first plane by about 90 degrees. The method includes concurrently abutting a base against the distal surface and the posterior surface, and further includes measuring a distance relative to the base. The measuring step includes spirally moving a first sleeve relative to a second sleeve.

The above-noted features and advantages of the present invention, as well as additional features and advantages, will be readily apparent to those skilled in the art upon reference to the following detailed description and the accompanying drawings, which include a disclosure of the best mode of making and using the invention presently contemplated.

### BRIEF DESCRIPTION OF THE DRAWINGS

FIG. 1 shows a perspective view of an exemplary apparatus according to the present invention;

FIG. 2 shows a partially exploded perspective view of the exemplary apparatus of FIG. 1;

FIG. 3 shows a plan view of the exemplary apparatus of FIG. 1;

FIG. 4 shows an exploded view of the axial distance scale of the exemplary apparatus of FIG. 1;

FIG. 5 shows a cross-sectional view of the exemplary apparatus of FIG. 1 (taken along line 5-5 of FIG. 1);

FIG. 6 shows an exploded perspective view of the anterior contact sub-assembly of the exemplary apparatus of FIG. 1;

FIG. 7 shows a plan view of exemplary operations of the exemplary apparatus of FIG. 1;

FIG. 8 shows a perspective view of an exemplary alternative apparatus according to the present invention;

FIG. 9 shows a plan view of exemplary operations of the exemplary alternative apparatus of FIG. 8;

FIG. 10 shows a perspective view of an additional exemplary alternative apparatus according to the present invention;

FIG. 11 shows a plan view of exemplary operations of the exemplary alternative apparatus of FIG. 10;

FIG. 12 shows a perspective view of an additional exemplary alternative apparatus according to the present invention; and

FIG. 13 shows a perspective view of an additional exemplary alternative apparatus according to the present invention.

### DETAILED DESCRIPTION OF EXEMPLARY EMBODIMENT(S)

Like reference numerals refer to like parts throughout the following description and the accompanying drawings. Additionally, unless otherwise stated all parts are made from one or more rigid sterilizable surgical materials such as stainless steel.

Further, as used herein the terms “medial,” “medially,” and the like mean pertaining to the middle, in or toward the middle, and/or nearer to the middle of the body when standing upright. Conversely, the terms “lateral,” “laterally,” and the like are used herein as opposed to medial. For example, the medial side of the knee is the side closest to the other knee and the closest sides of the knees are medially facing, whereas the lateral side of the knee is the outside of the knee and is laterally facing. Further, as used herein the term “superior” means closer to the top of the head and/or farther from the bottom of the feet when standing upright. Conversely, the term “inferior” is used herein as opposed to superior. For example, the heart is superior to the stomach and the superior surface of the tongue rests against the palate, whereas the stomach is inferior to the heart and the palate faces inferiorly toward the tongue. Also, as used herein the terms “anterior,” “anteriorly,” and the like mean nearer the front or facing away from the front of the body when standing upright, as opposed to “posterior,” “posteriorly,” and the like, which mean nearer the back or facing away from the back of the body.

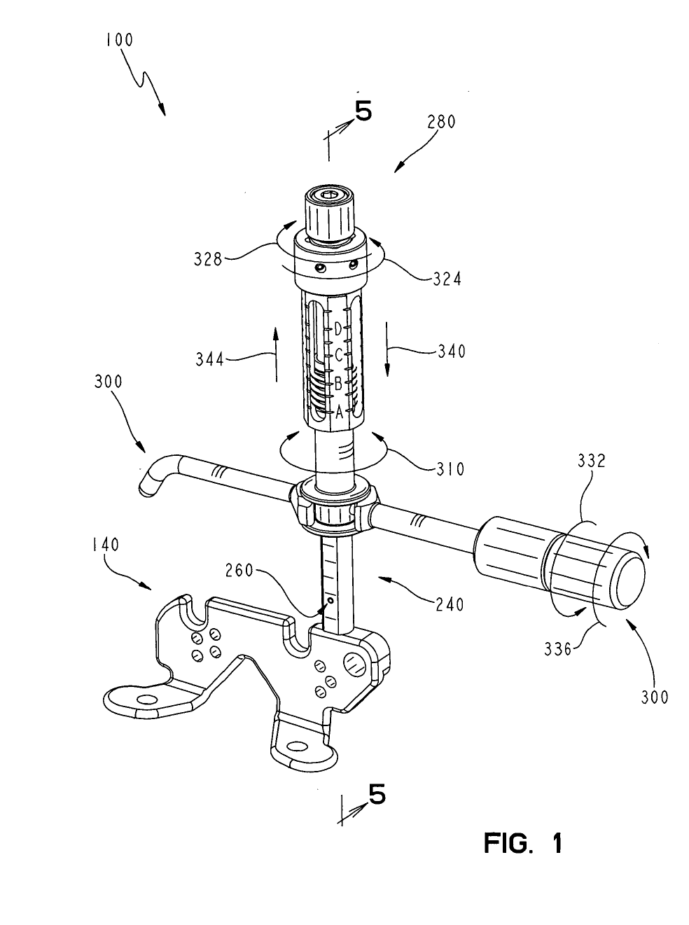

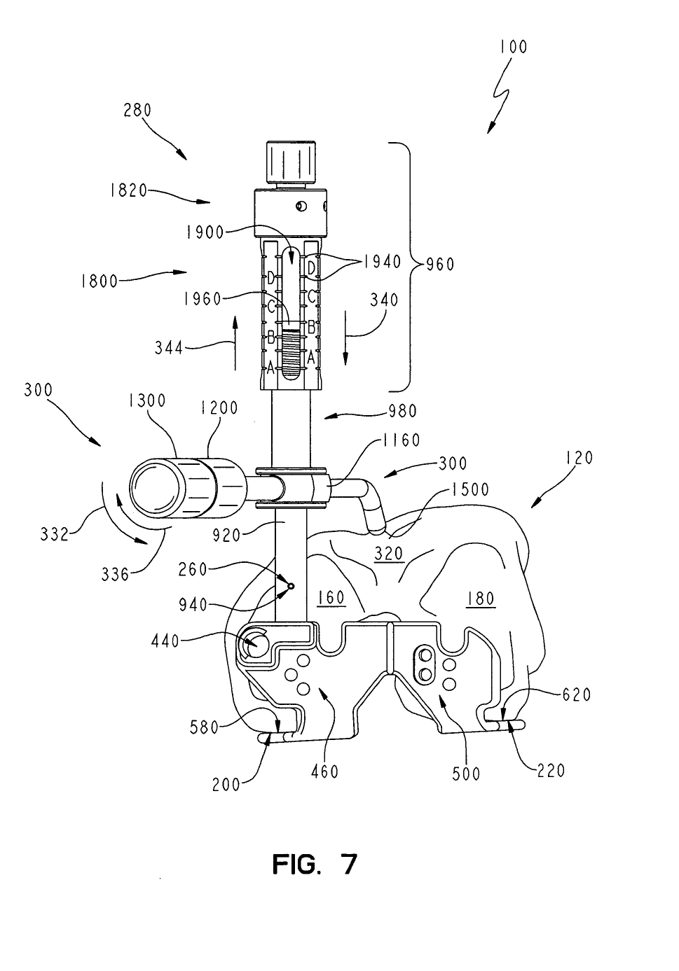

FIG. 1 shows a perspective view of an exemplary apparatus 100 according to the present invention. Apparatus 100 is configured to, among other things, size a typical distal femur 120 (see FIG. 7) as discussed further below. Apparatus 100 includes a base 140 configured to, among other things, concurrently abut a medial distal surface 160 (see FIG. 7) of distal femur 120, a lateral distal surface 180, a medial posterior surface 200, and a lateral posterior surface 220 as discussed further below.

Apparatus 100 further includes a rod 240 (FIG. 1) which extends from base 140 as discussed further below.

Apparatus 100 further includes a peg 260 configured to, among other things, help couple rod 240 to base 140.

Apparatus 100 further includes an axial distance scale 280 configured to, among other things, selectively axially expand or contract and to indicate a corresponding distal femoral size as discussed further below.

Apparatus 100 further includes an anterior contact sub-assembly 300 configured to, among other things, selectively rotate about scale 280 as indicated by directional lines 310 and to abut a desired anterior surface 320 (see FIG. 7) of distal femur 120 as discussed further below. Directional line 324 (FIG. 1), directional line 328, directional line 332, directional line 336, directional line 340, and directional line 344 are discussed further below in connection with exemplary operations of apparatus 100.

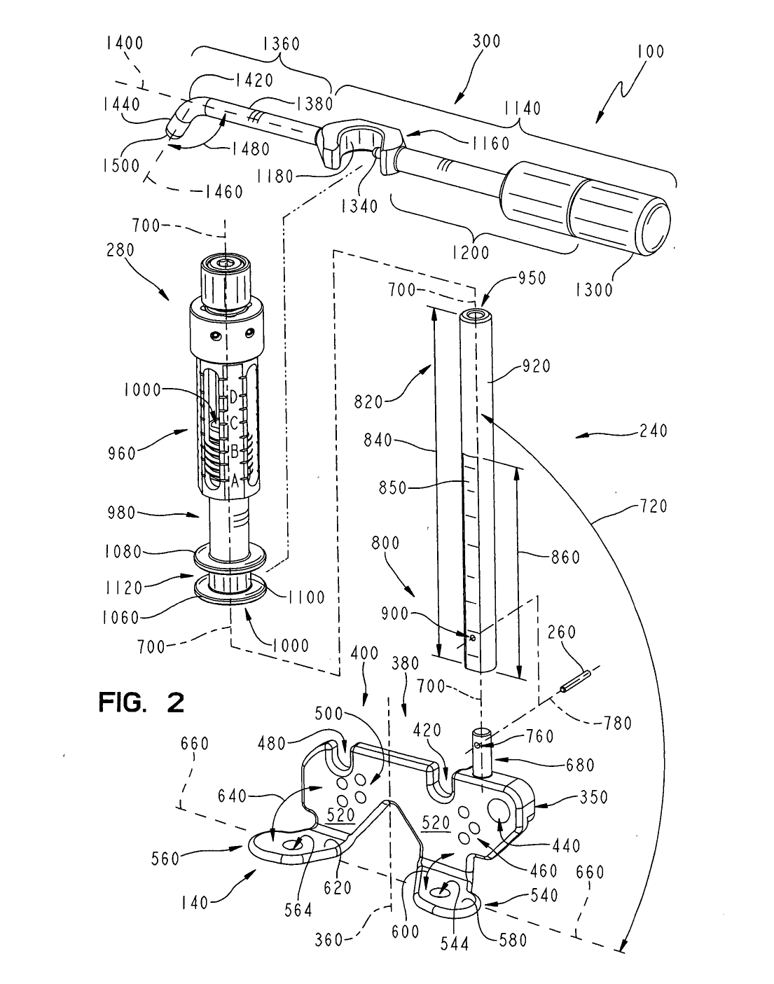

FIG. 2 shows a partially exploded perspective view of exemplary apparatus 100. As discernable in FIG. 2, base 140 is a bracket-like part including a wall 350. Relative to an imaginary dividing line 360, wall 350 includes a portion 380 and a portion 400. Portion 380 defines a generally U-shaped notch 420, a through-hole 440, and a plurality of through-holes 460. Meanwhile, portion 400 defines a generally U-shaped notch 480 and a plurality of through-holes 500. Further, wall 350 includes a substantially planar surface 520 extending over portion 380 and portion 400.

Base 140 also includes a tab 540 extending roughly orthogonally from portion 380 and a tab 560 extending roughly orthogonally from portion 400. Tab 540 defines a through-hole 544 and includes a substantially planar surface 580 surrounding through-hole 544. Surface 580 is angularly disposed from surface 520 by an angle 600. Tab 560 defines a through-hole 564 and includes a substantially planar surface 620 surrounding through-hole 564. Surface 620 is angularly disposed from surface 520 by an angle 640. In the exemplary embodiment, angle 600 is about 90 degrees and angle 640 is about 90 degrees. Additionally, surface 580 and surface 620 are coplanar along a line 660.

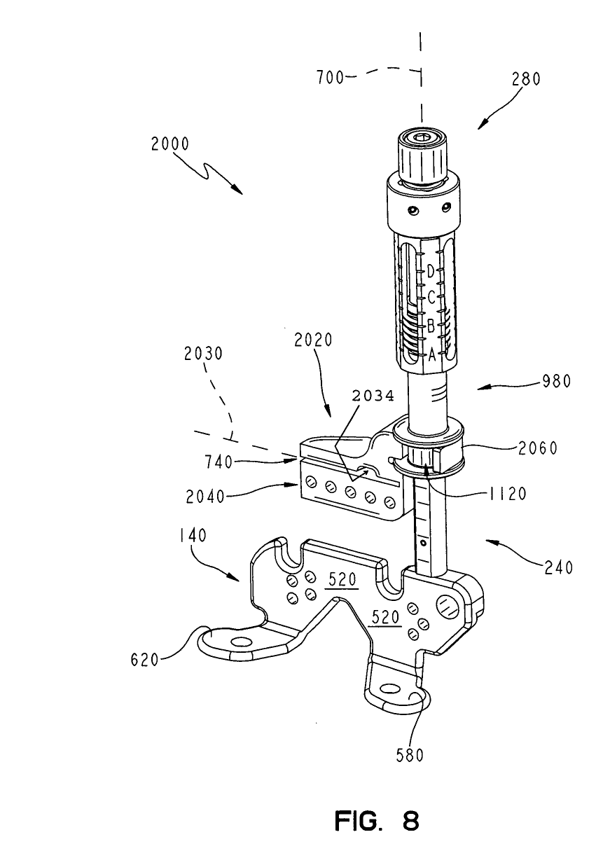

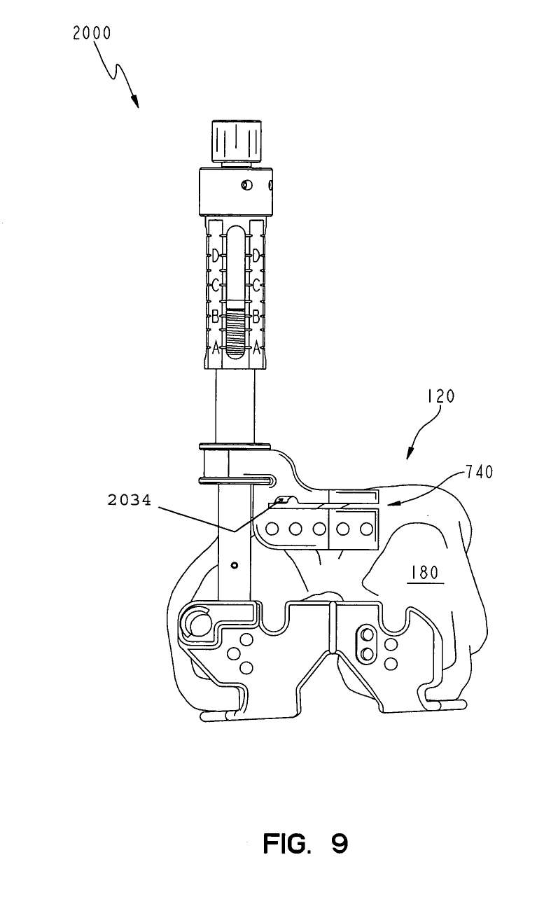

Base 140 also includes a generally cylindrical post 680 extending from portion 380 about an axis 700. Axis 700 is parallel to surface 520, and is angularly disposed from line 660 by a rotation angle 720 for positioning a cut slot 740 (see FIG. 8 and FIG. 9). In the exemplary embodiment, angle 720 is about 93 degrees. Further, post 660 defines a through-hole 760 extending about an axis 780 that is roughly perpendicular to axis 700.

As further discernable in FIG. 2, rod 240 extends longitudinally about axis 700. Also, rod 240 includes an end 800 and an opposing end 820, and has a longitudinal span 840. Rod 240 also includes a planar generally rectangular surface or flat 850. Flat 850 extends, generally in parallel with surface 520 (of base 140), from end 800 toward end 820.

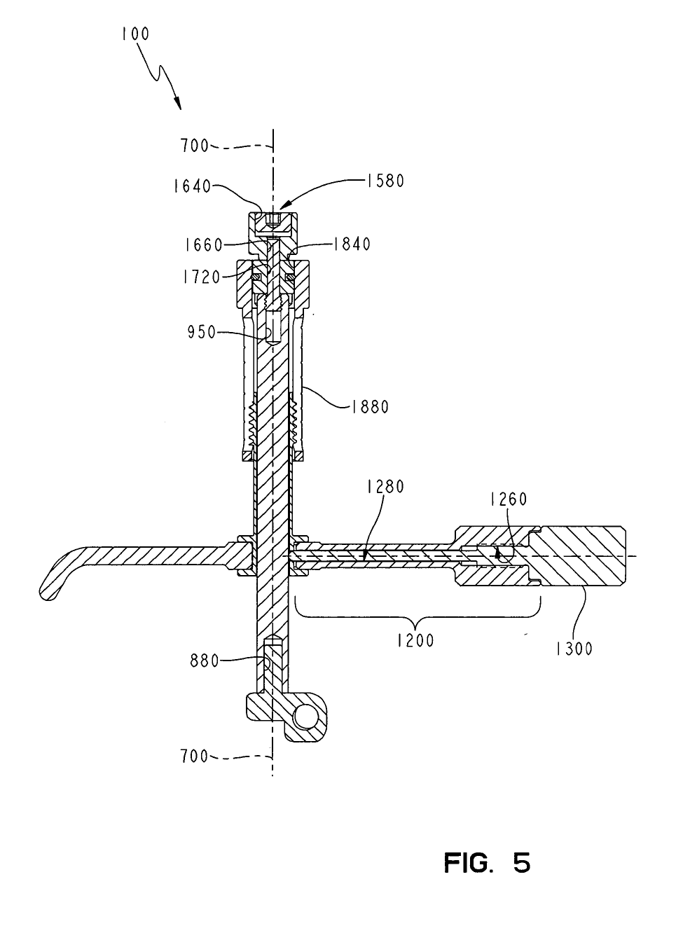

In the exemplary embodiment, flat 850 has a longitudinal span 860 that is about 60 percent of span 840. Meanwhile, end 800 defines a generally cylindrical socket 880 (see FIG. 5) opening from end 800 and extending axially inwardly toward end 820 along axis 700. End 800 also defines a through-hole 900 extending, coaxially with through-hole 760, through flat 850 into socket 880. Rod 240 also includes a radial sidewall surface 920 extending around flat 850. End 800 also defines a through-hole 940 (see FIG. 7) extending, coaxially with through-hole 760, through surface 920 into socket 880.

End 820 defines a generally cylindrical internally partially screw-threaded socket 950 (see also FIG. 5) opening from end 820 and extending axially inwardly toward end 800 along axis 700. When apparatus 100 is fully assembled, post 680 (of base 140) is press-fitted into socket 880 (of rod 240); additionally, peg 260 is inserted through through-hole 940 (of rod 240), through through-hole 760 (of post 680), and into through-hole 900 (of rod 240), where peg 260 is press-fitted and/or welded in place.

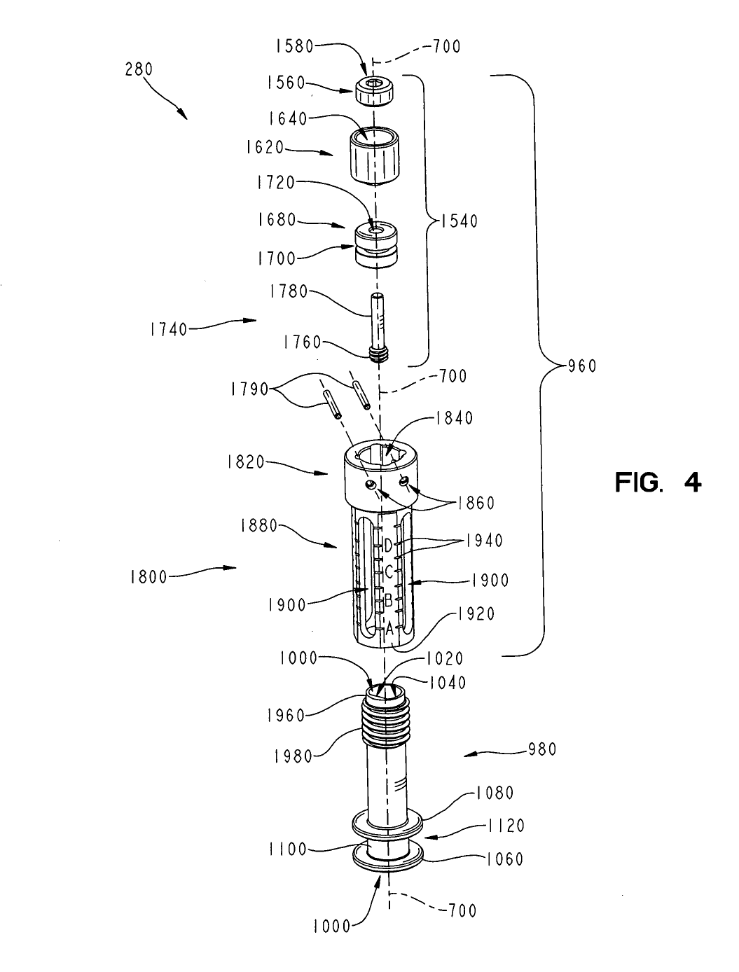

As further discernable in FIG. 2, scale 280 is longitudinally aligned about rod 240 along axis 700. Scale 280 includes an outer sub-assembly 960. Scale 280 also includes an inner sleeve 980 that defines a longitudinal through-channel 1000 including a generally planar surface or flat 1020 (see FIG. 4) and a substantially radial surface 1040 arching about flat 1020 (see FIG. 4).

When apparatus 100 is fully assembled, sleeve 980 is coaxially and movably spirally coupled into sub-assembly 960. Sleeve 980 also includes an annular wall 1060 and an annular wall 1080 that are axially spaced apart by an annular surface 1100. Wall 1060, wall 1080, and surface 1100 define an annular channel 1120.

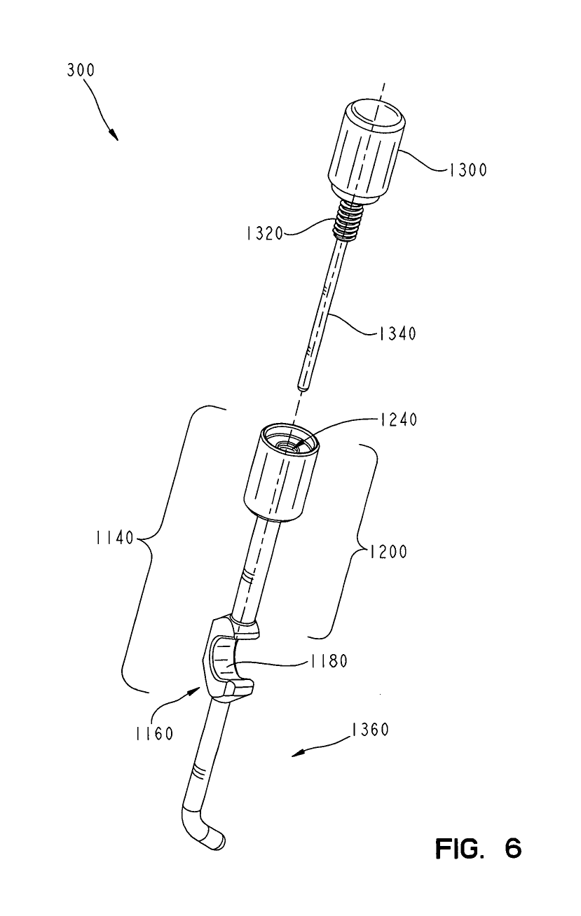

As further discernable in FIG. 2, sub-assembly 300 includes a clamp 1140. Clamp 1140 includes a generally C-shaped coupling member 1160. Member 1160 includes a generally radially inwardly facing surface 1180. Clamp 1140 also includes a handle 1200 extending from member 1160. Handle 1220 defines a longitudinal through-channel 1240 (see FIG. 6) including a screw-threaded portion 1260 (see FIG. 5) and a smooth portion 1280 (see FIG. 5).

Clamp 1140 also includes a generally cylindrical knob 1300 abutting handle 1200, a screw-threaded member 1320 (see FIG. 6) extending from knob 1300 into portion 1260 (see FIG. 5) of through-channel 1240 (see FIG. 6), and a coupling post 1340 (see also FIG. 6) extending from member 1320 (see FIG. 6) through portion 1280 (see FIG. 5) of through-channel 1240 (see FIG. 6) so as to protrude from surface 1180 of member 1160. Sub-assembly 300 also includes a probe 1360. Probe 1360 includes an elongated portion 1380 extending from clamp 1140 along a line 1400, further includes curved portion 1420 extending from portion 1380, further includes a straight portion 1440 extending from portion 1420 along a line 1460 that is disposed from line 1400 by an angle 1480, and further includes a blunt tip 1500.

In the exemplary embodiment angle 1480 is about 110 degrees. When apparatus 100 is fully assembled, member 1160 of clamp 1140 is positioned in channel 1120 (of sleeve 980) such that clamp 1140 removably and selectively rotatively couples sub-assembly 300 to sleeve 980 (and, thus, removably and selectively rotatively couples sub-assembly 300 to scale 280) and line 1400 is roughly perpendicular to axis 700.

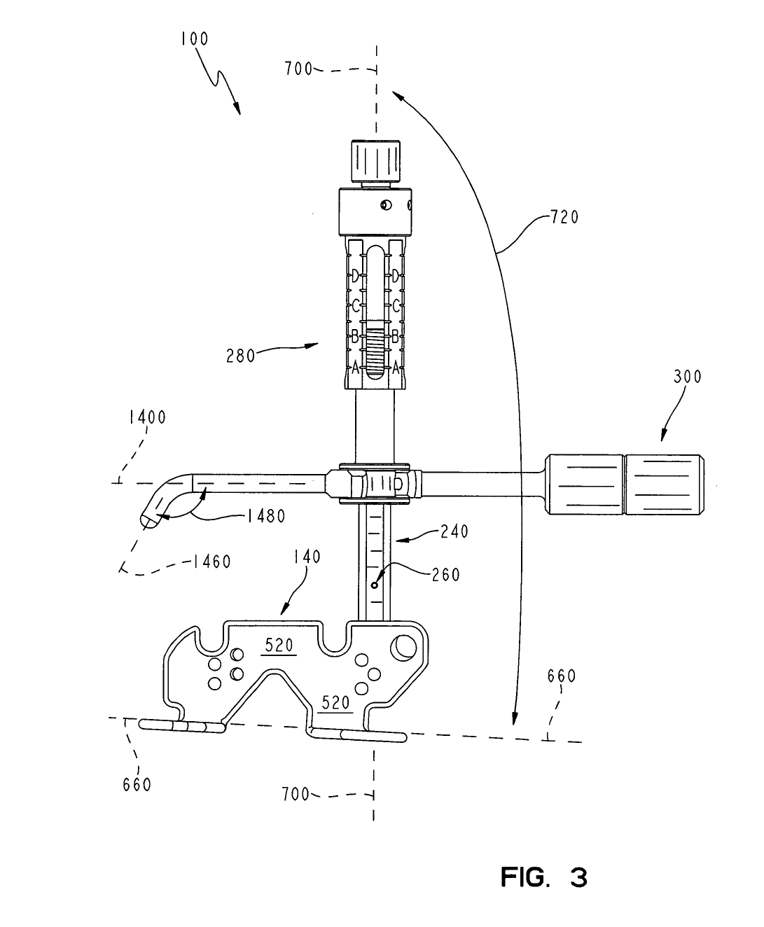

FIG. 3 shows a plan view of apparatus 100. Base 140 (including surface 520), rod 240, peg 260, scale 280, sub-assembly 300, line 660, axis 700, angle 720, line 1400, line 1460, and angle 1480, among other things, are all at least partially discernable in FIG. 3.

FIG. 4 shows an exploded view of scale 280. As discernable in FIG. 4, sub-assembly 960 includes a bearing sub-assembly 1540. Sub-assembly 1540 includes a disk-like member 1560 defining a hex socket 1580. Sub-assembly 1540 also includes a collar-like member 1620. Member 1620 defines a cylindrical socket 1640 (see also FIG. 5) and a cylindrical through-channel 1660 (see FIG. 5) extending coaxially from socket 1640 (see FIG. 5). Member 1560 is inserted into socket 1640 and welded into place. Sub-assembly 1540 also includes a bearing 1680. Bearing 1680 defines an annular channel or race 1700 and a cylindrical through-channel 1720.

Sub-assembly 1540 also includes a peg 1740. Peg 1740 includes a generally cylindrical screw-threaded portion 1760 and a cylindrical screw-threaded portion 1780. Portion 1780 is inserted through through-channel 1720 (of bearing 1680) and welded into through-channel 1660 (of member 1620) (see also FIG. 5). Sub-assembly 960 also includes a pair of identically configured pegs 1790. Further, sub-assembly 960 includes a graduated sleeve 1800.

Sleeve 1800 includes an annular knob 1820 defining a generally cylindrical through-channel 1840 (see also FIG. 5) and defining a pair of through-channels 1860 extending roughly perpendicularly to through-channel 1840 and positioned to align roughly tangentially with race 1700 upon full assembly of sub-assembly 960. Sleeve 1800 also includes a partially internally spirally-threaded sidewall 1880 (see also FIG. 5) extending longitudinally from knob 1820.

Sidewall 1880 defines a plurality of elongated apertures 1900 and includes an outer surface 1920 with a plurality of femoral implant size graduations 1940 thereon. For full assembly of sub-assembly 960, sub-assembly 1540 is coupled to sleeve 1800 by inserting sub-assembly 1540 into through-channel 1840 (of sleeve 1800), by positioning bearing 1680 such that through-channels 1860 are aligned roughly tangentially with race 1700, by inserting pegs 1790 through the respective through-channels 1860, and by press-fitting and/or welding pegs 1790 in place.

As further discernable in FIG. 4, sleeve 980 also includes an end portion 1960 and external spiral threads 1980 extending from portion 1960 toward wall 1080. Axis 700, through-channel 1000, flat 1020, surface 1040, wall 1060, wall 1080, surface 1100, and channel 1120 (of sleeve 980), among other things, are all at least partially discernable in FIG. 4.

Here, it is noted that in assembly of apparatus 100, sleeve 980 is first separated from sub-assembly 960. Next, end 800 of rod 240 is inserted into through-channel 1000 (of sleeve 980). Next, end 820 of rod 240 is inserted into through-channel 1840 (of sleeve 1800 of sub-assembly 960), and peg 1740 is screwed tightly into socket 950 (of end 820) by torquing an Allen wrench or any other suitable tool in socket 1580.

Thus, sleeve 1800 is axially fixed yet rotatively movably coupled to end 820 of rod 240. Next, sleeve 980 is spiraled into sleeve 1800 of sub-assembly 960 until end 800 of rod 240 protrudes from sleeve 980 and through-hole 900 is exposed. Thus, sleeve 980 is spirally movably coupled to sleeve 1800. Next, post 680 (of base 140) is press-fitted into socket 880 (of rod 240).

Next, peg 260 is inserted through through-hole 940 (of rod 240), through through-hole 760 (of post 680), and into through-hole 900 (of rod 240), where peg 260 is press-fitted and/or welded in place. Lastly, member 1160 of clamp 1140 is positioned in channel 1120 (of sleeve 980) such that clamp 1140 removably and selectively rotatively couples sub-assembly 300 to sleeve 980 (and, thus, removably and selectively rotatively couples sub-assembly 300 to scale 280) and line 1400 is roughly perpendicular to axis 700.

FIG. 5 shows a cross-sectional view of apparatus 100 (taken along line 5-5 of FIG. 1). Axis 700, socket 880, socket 950, handle 1200, through-channel 1260, through-channel 1280, knob 1300, socket 1640, through-channel 1660, through-channel 1720, through-channel 1840, and sidewall 1880, among other things, are all at least partially discernable in FIG. 5.

FIG. 6 shows an exploded perspective view of sub-assembly 300. Clamp 1140 (including member 1160, surface 1180, handle 1200, and through-channel 1240), knob 1300, member 1320, post 1340, and probe 1360, among other things, are all at least partially discernable in FIG. 6.

FIG. 7 shows a plan view of exemplary operations of apparatus 100. In operation, a surgeon or other user axially contracts scale 280 (to provide sufficient clearance between tip 1500 and surface 580 and surface 620 for receiving distal femur 120) by rotating knob 1820 as indicated by directional line 324 (see FIG. 1) such that sub-assembly 960 draws sleeve 980 into sleeve 1800 (see FIG. 4) as indicated by directional line 344 (see also FIG. 1).

Next, the user abuts surface 520 (see FIG. 2 and FIG. 3) against a medial distal surface 160 of distal femur 120 and concurrently abuts surface 520 against a lateral distal surface 180 of distal femur 120. Further, and concurrently, the user abuts surface 580 against a medial posterior surface 200 of distal femur 120, and abuts surface 620 against a lateral posterior surface 220 of distal femur 120. The user may temporarily fix apparatus 100 to distal femur 120 by driving one or more bone pins or other suitable fasteners (not shown) through any of through-hole 440, through-holes 460, through-holes 500, through-hole 544, and/or through-hole 564. However, as shown in FIG. 7, such fasteners are preferably omitted.

Next, the user turns knob 1300 as indicated by directional line 332 (see also FIG. 1) so as to retract post 1340 (see FIG. 2 and FIG. 6) sufficiently from member 1160 to free up or release sub-assembly 300 for rotation about sleeve 980 as indicated by directional lines 310 (see FIG. 1). The user visually locates the desired anterior surface 320 of distal femur 120 and then maneuvers handle 1200 to rotate sub-assembly 300 about sleeve 980 as indicated by directional lines 310 (see FIG. 1) such that tip 1500 is preliminarily rotationally positioned roughly superiorly to surface 320. Preferably, surface 320 is the anterior cortex (i.e., the natural depression or valley between condyles).

However, it is noted that surface 320 may correspond to any other suitable location on distal femur 120.

After preliminarily rotationally positioning tip 1500, the user axially expands scale 280 by rotating knob 1820 as indicated by directional line 328 (see FIG. 1) such that sub-assembly 960 extends sleeve 980 from sleeve 1800 (see FIG. 4) as indicated by directional line 340 (see also FIG. 1). It should be appreciated that this causes tip 1500 to move toward surface 320. However, it is noted that abutment of flat 850 (see FIG. 2) and flat 1020 (see FIG. 4) prevents rotation of sleeve 980. As tip 1500 approaches surface 320, the user further maneuvers handle 1200 to rotate sub-assembly 300 about sleeve 980 as indicated by directional lines 310 (see FIG. 1) such that tip 1500 is finally rotationally positioned superiorly to surface 320.

After finally rotationally positioning tip 1500, the user turns knob 1300 as indicated by directional line 336 (see also FIG. 1) so as to extend post 1340 (see FIG. 2 and FIG. 6) sufficiently from member 1160 to rotatively fix sub-assembly 300 about sleeve 980.

After rotatively fixing sub-assembly 300 about sleeve 980, the user resumes and/or continues the axially expansion of scale 280 until tip 1500 desirably firmly abuts surface 320. This provides a final setting for scale 280. The resulting axial position of portion 1960 of sleeve 980 relative to graduations 1940 of sleeve 1800 indicates a corresponding anterior-posterior dimension or femoral implant size. Accordingly, the user visually observes portion 1960 through one or more of apertures 1900 (see also FIG. 4) and reads the size from graduations 1940. Here, it is noted that the spirally movable coupling of sleeve 980 to sleeve 1800 provides a practically infinite axial adjustment resolution for scale 280. Additionally, it is noted that this configuration resists slippage or shifts in the final setting for scale 280 without additional undesirably cumbersome and/or complex locking mechanisms.

FIG. 8 shows a perspective view of an exemplary alternative apparatus 2000 according to the present invention. Apparatus 2000 is like apparatus 100 with the exception that sub-assembly 300 is replaced by a cut guide 2020. Guide 2020 is configured to, among other things, guide a saw blade or other suitable resection tool. Guide 2020 defines cut slot 740. Slot 740 is roughly orthogonal to surface 520 and extends generally away from sleeve 980 and rod 240 along a line 2030 that is roughly perpendicular to axis 700. Guide 2020 also defines a through-channel 2034 that is contiguous with slot 740. Guide 2020 also defines a plurality of through-channels 2040. Guide 2020 also includes a hook-like arm 2060 removably positioned on sleeve 980 in channel 1120.

FIG. 9 shows a plan view of exemplary operations of apparatus 2000. For operation of apparatus 2000, the user first makes a final setting for scale 280 via operation of apparatus 100 as discussed above. After setting scale 280 with apparatus 100 (and preferably without removing surface 520, surface 580, or surface 620 from contact with distal femur 120 or otherwise significantly moving base 140 relative to distal femur 120), the user constructs apparatus 2000 by replacing sub-assembly 300 with guide 2020. Next, the user may optionally insert a standard 1/8 inch drill bit (not shown) into through-channel 2034 and drive it through distal femur 120 to pre-check where a saw blade or other suitable resection tool (not shown) would exit distal femur 120 if guided through slot 740. The user may then adjust or fine tune scale 280 to a more desirable setting by turning knob 1820 accordingly. Such verification and adjustment procedures may help to avoid undesirably jagged or stepped exiting of the saw blade from distal femur 120. Undesirably jagged or stepped existing of the saw blade produces what is commonly referred to in the art as a “notched” cut. In any event, the user guides a saw blade or other suitable resection tool (not shown) in slot 740 to make a corresponding anterior cut to distal femur 120.

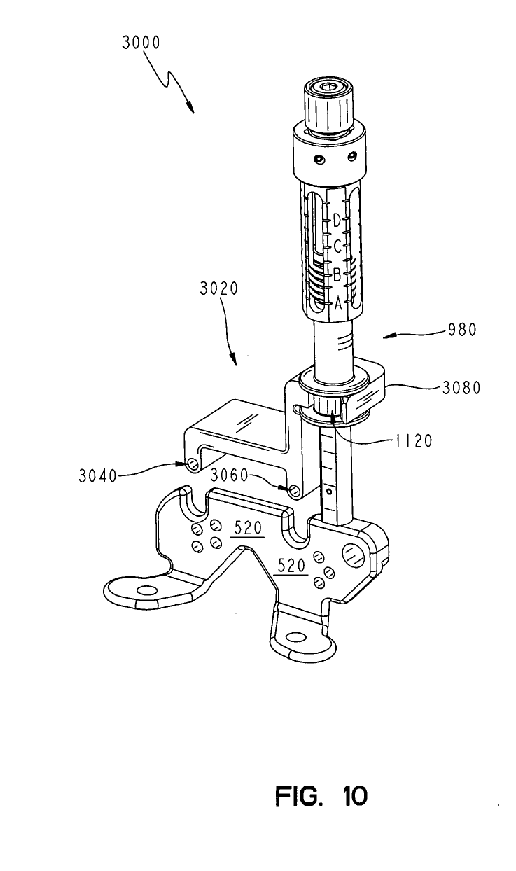

FIG. 10 shows a perspective view of an additional exemplary alternative apparatus 3000 according to the present invention. Apparatus 3000 is like apparatus 100 with the exception that sub-assembly 300 is replaced by a bone pin guide 3020. Guide 3020 is configured to, among other things, guide placement of one or more bone pins or similar devices. Guide 3020 defines a pin slot 3040 and a pin slot 3060. Slot 3040 and slot 3060 are each roughly orthogonal to surface 520. Guide 3020 also includes a hook-like arm 3080 removably positioned on sleeve 980 in channel 1120.

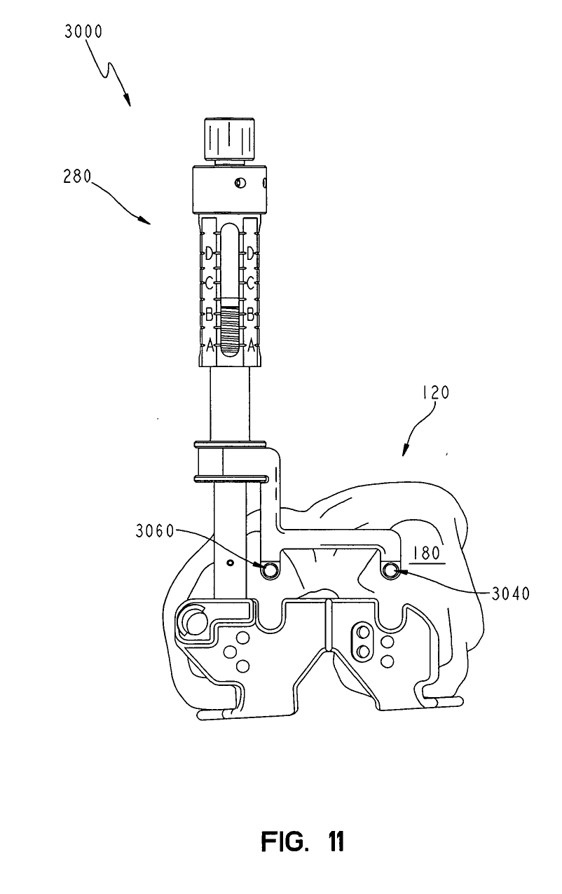

FIG. 11 shows a plan view of exemplary operations of apparatus 3000. For operation of apparatus 3000, the user first makes a final setting for scale 280 via operation of apparatus 100 as discussed above. After setting scale 280 with apparatus 100 (and preferably without removing surface 520, surface 580, or surface 620 from contact with distal femur 120 or otherwise significantly moving base 140 relative to distal femur 120), the user constructs apparatus 3000 by replacing sub-assembly 300 with guide 3020. Then, the user guides one or more bone pins or similar devices (not shown) in slot 3040 and/or slot 3060 and drives them into distal femur 120. It should be appreciated that the user may then suitably alignment additional surgical instrument (not shown) with the bone pins as desired.

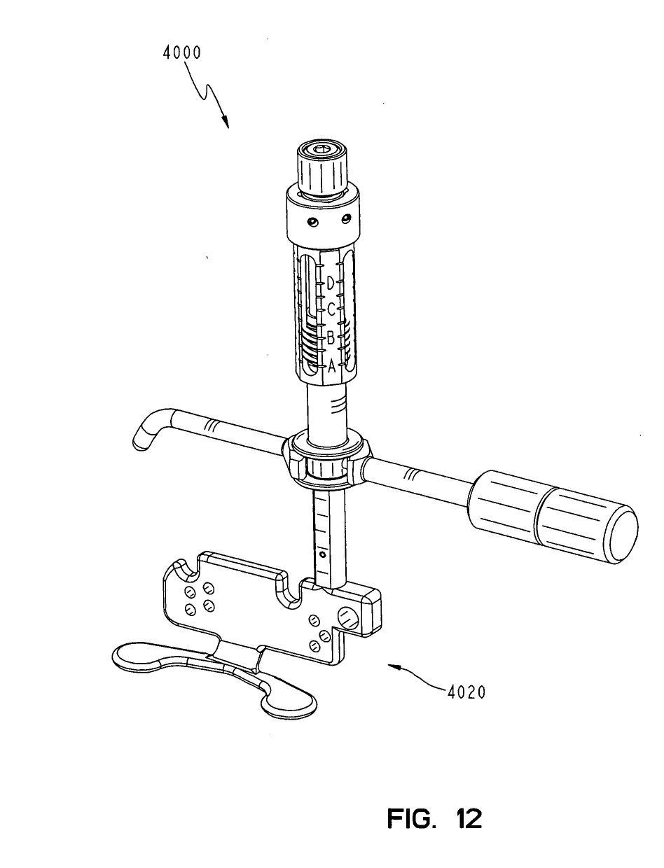

FIG. 12 shows a perspective view of an additional exemplary alternative apparatus 4000 according to the present invention. Apparatus 4000 is made and used like apparatus 100 with the exception that base 140 is replaced by an alternative base 4020.

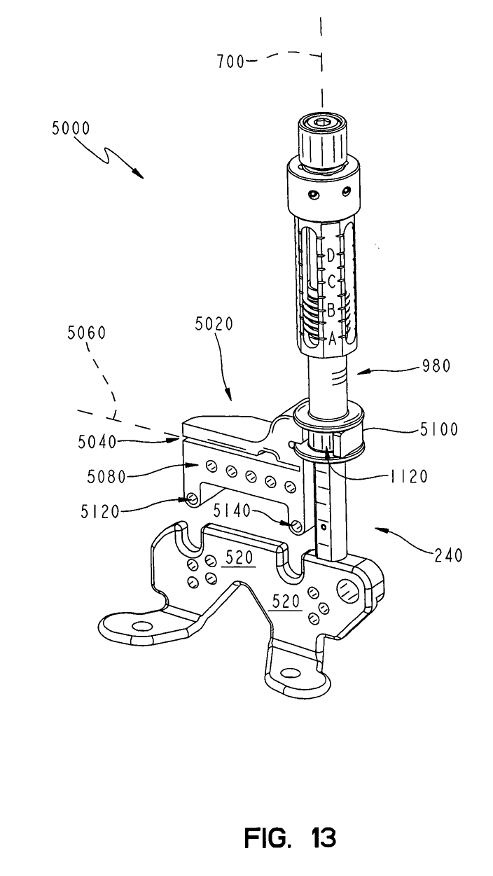

FIG. 13 shows a perspective view of an additional exemplary alternative apparatus 5000 according to the present invention. Apparatus 5000 is made and used like apparatus 2000 and/or apparatus 3000 with the exception that guide 2020 and/or guide 3020, respectively, is replaced by an alternative combination cut/pin guide 5020. Guide 5020 is configured to, among other things, guide a saw blade or other suitable resection tool and to guide placement of one or more bone pins or similar devices.

Guide 5020 defines cut slot 5040 roughly orthogonal to surface 520 and extends generally away from sleeve 980 and rod 240 along a line 5060 that is roughly perpendicular to axis 700. Guide 5020 also defines a plurality of through-channels 5080. Guide 5020 also includes a hook-like arm 5100 removably positioned on sleeve 980 in channel 1120. Guide 5020 also defines a pin slot 5120 and a pin slot 5140. Slot 5120 and slot 5140 are each roughly orthogonal to surface 520.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. An apparatus for sizing a distal femur having a distal surface located in a first plane and a posterior surface located in a second plane angularly disposed from the first plane by about 90 degrees, the apparatus comprising:
a base configured to concurrently abut the distal surface and the posterior surface; and
an axial distance scale including a first sleeve axially fixedly coupled to the base and further including a second sleeve spirally movably coupled to the first sleeve.

2. The apparatus of claim 1, wherein the first sleeve is rotatively coupled to the base.

3. The apparatus of claim 1, further comprising an elongated member selectively rotatable about the second sleeve.

4. The apparatus of claim 3, wherein the elongated member includes a clamp and the second sleeve extends through the clamp.

5. The apparatus of claim 4, wherein the second sleeve defines an annular channel and the clamp is positioned in the channel.

6. The apparatus of claim 1, further comprising a rod including a first end fixedly coupled to the base and further including a second end axially fixedly coupled to the first sleeve.

7. The apparatus of claim 6, wherein the rod extends through the second sleeve.

8. The apparatus of claim 6, wherein the second end of the rod is rotatively coupled to the first sleeve.

9. The apparatus of claim 8, further comprising:
a bearing fixedly coupled to the second end of the rod; and
at least one peg coupling the bearing to the first sleeve.

10. The apparatus of claim 9, wherein the rod extends through the second sleeve.

11. The apparatus of claim 10, further comprising an elongated member selectively rotatable about the second sleeve.

12. The apparatus of claim 11, wherein the elongated member includes a clamp and the second sleeve extends through the clamp.

13. The apparatus of claim 12, wherein the second sleeve defines an annular channel and the clamp is positioned in the channel.

14. The apparatus of claim 10, further comprising a cut guide including a portion positioned on the second sleeve.

15. The apparatus of claim 14, wherein the second sleeve defines an annular channel, and the portion of the cut guide includes a hook-like member positioned in the channel.

16. The apparatus of claim 15, wherein the cut guide defines a bone pin guide slot.

17. The apparatus of claim 15, wherein the cut guide defines a cut slot and further defines a through-channel contiguous with the cut slot.

18. The apparatus of claim 10, further comprising a pin guide including a portion positioned on the second sleeve.

19. The apparatus of claim 18, wherein the second sleeve defines an annular channel, and the portion of the pin guide includes a hook-like member positioned in the channel.

20. The apparatus of claim 1, further comprising a cut guide including a portion positioned on the second sleeve.

21. The apparatus of claim 20, wherein the second sleeve defines an annular channel, and the portion of the cut guide includes a hook-like member positioned in the channel.

22. The apparatus of claim 21, wherein the cut guide defines a bone pin guide slot.

23. The apparatus of claim 21, wherein the cut guide defines a cut slot and further defines a through-channel contiguous with the cut slot.

24. The apparatus of claim 1, further comprising a pin guide including a portion positioned on the second sleeve.

25. The apparatus of claim 24, wherein the second sleeve defines an annular channel, and the portion of the pin guide includes a hook-like member positioned in the channel.

26. An apparatus for sizing a distal femur having a distal surface located in a first plane and a posterior surface located in a second plane angularly disposed from the first plane by about 90 degrees, the apparatus comprising:
means for concurrently abutting the distal surface and the posterior surface; and
means, coupled to the abutting means, for measuring a distance relative to the abutting means.

27. The apparatus of claim 26, further comprising means, removably coupled to the measuring means, for guiding a saw blade.

28. The apparatus of claim 26, further comprising means, removably coupled to the measuring means, for guiding a bone pin.

29. The apparatus of claim 28, further comprising means, integrated with the pin guiding means, for guiding a saw blade.

30. A method for sizing a distal femur having a distal surface located in a first plane and a posterior surface located in a second plane angularly disposed from the first plane by about 90 degrees, the method comprising the steps of:
concurrently abutting a base against the distal surface and the posterior surface; and
measuring a distance relative to the base;
wherein the measuring step includes spirally moving a first sleeve relative to a second sleeve.

31. The method of claim 30, further comprising the steps of:
rotating an elongated member on the first sleeve; and
abutting the elongated member against the distal femur during the step of concurrently abutting the base against the distal surface and the posterior surface.

32. The method of claim 31, further comprising the step of rotatively fixing the elongated member to the first sleeve.

33. The method of claim 32, further comprising the step of clamping the elongated member to the first sleeve.

34. The method of claim 33, further comprising the step of hooking a cut guide onto the first sleeve.

35. The method of claim 33, further comprising the step of hooking a pin guide onto the first sleeve.

36. The method of claim 30, further comprising the step of hooking a cut guide onto the first sleeve.

37. The method of claim 30, further comprising the step of hooking a pin guide onto the first sleeve.
